(19) United States
(12) Patent Application Publication
(10) Pub. No.: US 2001/0005764 A1
(43) Pub. Date: Jun. 28, 2001

(54) Process for the preparation of 2-aminomethyl-1,5-pentanediamine

(76) Inventors: Konrad Fischer, Odenthal (DE); Frank Richter, Leverkusen (DE); Anatoly Bazanov, St. Petersburg (RU); Alexandre Timofeev, St. Petersburg (RU); Natalja Zubritskaya, St. Petersburg (RU); Galina Smirnova, St. Petersburg (RU)

Correspondence Address: Bayer Corportation, Patent Department, 100 Bayer Road, Pittsburgh, PA, 15205-9741, US

(21) Appl. No.: 09/496,067

(22) Filed: Feb. 1, 2000

(30) Foreign Application Priority Data
Feb. 9, 1999 (DE) 19905277.8

(51) Int. Cl. C07C 209/00

(52) U.S. Cl. 564/491; 564/489; 564/469

(57) ABSTRACT

The present invention relates to a process for the preparation of 2-aminomethyl-1,5-pentanediamine by simultaneously reacting 2,4-dicyano-1-butene, hydrogen and ammonia in one step without isolation of 2-amino-methyl-1,5-pentanedinitrile; the products are useful as an intermediate in the production of coating compositions, surfactants, detergents and as a component of epoxide compositions.

### BACKGROUND OF THE INVENTION

1. Field of the Invention

The present invention relates to a process for the preparation of 2-aminomethyl-1,5-pentanediamine, which can be used as an intermediate in the production of coating compositions, surfactants, detergents and as a component of epoxide compositions.

2. Description of the Invention

The production of 2-aminomethyl-1,5-pentanediamine by the hydrogenation of 2-aminomethyl-1,5-pentanedinitrile in an ammoniacal medium is described in Jap. Pat. 46-2484 (1971). The main disadvantage of this process is that it uses 2-aminomethyl-1,5-pentanedinitrile as a starting material, which has to be prepared by a complicated process of synthesis and isolation. Also 2-aminomethyl-1,5-pentanedinitrile, which is obtained, for example, by the addition of ammonia to 2,4-dicyano-1-butene (dimeric acrylonitrile), has a very high boiling point and is stable only to a limited extent. Its isolation and handling are greatly hampered because of this and product losses and a high consumption of energy during its production and processing are unavoidable.

An object of the present invention is to provide a more economically efficient process for the industrial production of 2-aminomethyl-1,5-pentanediamine.

This object may be obtained with the process according to the present invention as set forth hereinafter by the catalytic reaction of 2,4-dicyano-1-butene with ammonia and hydrogen in a one-step reaction without isolation of 2-aminomethyl-1,5-pentanedinitrile.

### SUMMARY OF THE INVENTION

The present invention relates to a process for the preparation of 2-aminomethyl-1,5-pentanediamine by simultaneously reacting 2,4-dicyano-1-butene, hydrogen and ammonia in one step without isolation of 2-aminomethyl-1,5-pentanedinitrile.

### DETAILED DESCRIPTION OF THE INVENTION

The reaction according to the invention takes place as follows:

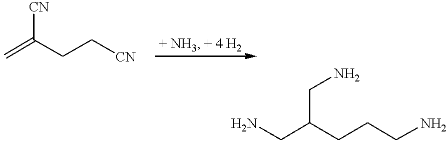

The process for preparing 2-aminomethyl-1,5-pentanediamine by the reaction of 2,4-dicyano-1-butene, hydrogen and ammonia preferably takes place in a fixed bed tubular reactor filled with the hydrogenation catalyst. The starting materials are preferably mixed beforehand in a continuous flow tank and cobalt- and/or nickel-containing catalysts are used as the hydrogenation catalysts.

2,4-dicyano-1-butene can undergo reaction with or without the use of solvents. Suitable solvents include aliphatic alcohols having 1 to 6 carbon atoms, preferably 1 to 4 carbon atoms.

The reaction is carried out at a temperature of 20 to 200° C., preferably 40 to 160° C. and more preferably 50 to 100° C. The hydrogenation pressure is 50 to 250 atm., preferably 100 to 220 atm.

Compared with the known prior art processes, the process according to the invention for the production of 2-aminomethyl-1,5-pentanediamine from 2,4-dicyano-1-butene, hydrogen and ammonia has the advantage that it can be carried out in one step, starting from inexpensive starting materials and without the costly isolation and purification of intermediate products.

The catalytic fixed bed reactor with stationary hydrogenation catalyst may optionally be operated continuously and the reaction components may be continuously mixed beforehand in a continuous-flow tank. The reaction mixture leaving the reactor is cooled to ambient temperature, the excess hydrogen-ammonia gaseous mixture is separated off and returned to the process, then ammonia and any solvents optionally used are removed from the liquid reaction products by distillation and the residue is rectified. The primary by-products are unreacted 2,4-dicyano-1-butene, 3-aminomethyl-piperidine and 2-methyl-1,5-pentanediamine. The resulting product contains, based on analysis by gas chromatography, at least 95 wt. %, preferably more than 98 wt. %, of 2-aminomethyl-1,5-pentanediamine.

Preferably, cobalt catalysts doped with an alkali metal oxide are used to ensure that the catalyst has a long useful life with consistently high activity and, in particular, selectivity towards the formation of 2-aminomethyl-1,5-pentanediamine. It is particularly preferred to use cobalt catalysts which have been doped with an alkali metal oxide as a surface additive. The quantity of additive is 1 to 15 wt. %, preferably 1 to 10 wt. %, based on the total quantity of catalyst.

The invention is further illustrated but is not intended to be limited by the following examples in which all parts and percentages are by weight unless otherwise specified.

### EXAMPLES

### Example 1

A tubular reactor was loaded with 200 cm³ of a granulated cobalt catalyst which contained 65% Co, 3.5% Mn and 3% H₃PO₄ and the catalyst was reduced. Then 10 ml of a 50% solution of 2,4-dicyano-1-butene in 2-propanol and 60 ml of liquid ammonia were introduced at 25° C. into the lower part of a mixing tube having a volume of 500 cm³ and hydrogen was introduced at a rate of 100 l/h. The resulting gas-liquid mixture was passed into the top of the reactor at 80° C. and 200 atm. The reaction mixture was withdrawn from the lower part of the reactor and cooled, the hydrogen-ammonia gaseous mixture was separated off and ammonia was removed from the liquid products. Then 2-propanol was distilled off under vacuum and the residue was rectified in order to remove impurities and unreacted 2-methyl-1,5-pentanediamine, 3-aminomethylpiperidine and 2,4-dicyano-1-butene from the crude triamine. The product was collected as the fraction having a boiling point of 115 to 117° C. (3 to 4 mm). In this experiment, the yield of 2-aminomethyl-1,5-pentanediamine was 63%, based on the reacted 2,4-dicyano-1-butene.

### Example 2

10% sodium oxide was added as surface-modifying additive to a catalyst of the type described in 1 above. 200 cm³ of this catalyst was placed in the reactor and the procedure described in Example 1 was repeated. 1.9 g of 2-aminomethyl-1,5-pentanediamine having a purity of 98% was obtained. The yield, based on the amount of 2,4-dicyano-1-butene that reacted, was 66.6%.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the preparation of 2-aminomethyl-1,5-pentanediamine by simultaneously reacting 2,4-dicyano-1-butene, hydrogen and ammonia in one step without isolation of 2-aminomethyl-1,5-pentanedinitrile.

2. The process of claim 1 which comprises carrying out the reaction in a fixed bed tubular catalytic reactor filled with a hydrogenation catalyst at a temperature of 20 to 200° C. and a pressure of 50 to 250 atm.

3. The process of claim 2 which comprises mixing the reactants in a continuous flow tank before they are introduced into the fixed bed tubular catalytic reactor.

4. The process of claim 2 wherein the hydrogenation catalyst comprises a cobalt- and/or nickel-containing catalyst.

5. The process of claim 2 which comprises carrying out the reaction at a temperature of 50 to 100° C.

6. The process of claim 4 which comprises carrying out the reaction at a temperature of 50 to 100° C.

7. The process of claim 2 which comprises carrying out the reaction at a pressure of 100 to 220 atm.

8. The process of claim 4 which comprises carrying out the reaction at a pressure of 100 to 220 atm.

9. The process of claim 5 which comprises carrying out the reaction at a pressure of 100 to 220 atm.

10. The process of claim 6 which comprises carrying out the reaction at a pressure of 100 to 220 atm.

11. The process of claim 4 wherein the cobalt- and/or nickel-containing catalyst has been doped with 1 to 15 wt. %, based on the weight of the catalyst, of an alkali metal oxide and/or alkaline-earth metal oxide.

12. The process of claim 6 wherein the cobalt- and/or nickel-containing catalyst has been doped with 1 to 15 wt. %, based on the weight of the catalyst, of an alkali metal oxide and/or alkaline-earth metal oxide.

13. The process of claim 8 wherein the cobalt- and/or nickel-containing catalyst has been doped with 1 to 15 wt. %, based on the weight of the catalyst, of an alkali metal oxide and/or alkaline-earth metal oxide.

14. The process of claim 10 wherein the cobalt- and/or nickel-containing catalyst has been doped with 1 to 15 wt. %, based on the weight of the catalyst, of an alkali metal oxide and/or alkaline-earth metal oxide.

15. The process of claim 11 wherein the cobalt- and/or nickel-containing catalyst has been doped with sodium oxide.

16. The process of claim 12 wherein the cobalt- and/or nickel-containing catalyst has been doped with sodium oxide.

17. The process of claim 13 wherein the cobalt- and/or nickel-containing catalyst has been doped with sodium oxide.

18. The process of claim 14 wherein the cobalt- and/or nickel-containing catalyst has been doped with sodium oxide.
